(19) United States
(12) Patent Application Publication
(10) Pub. No.: US 2010/0112702 A1
(43) Pub. Date: May 6, 2010

(54) Method designed to divert glucose away from the glycolytic pathway

(76) Inventor: Benjamin Y. Klein, New York, NY (US)

Correspondence Address: Benjamin Y. Klein, 390 Fort Washington Ave., Apt. 4E, New York, NY, 10033, US

(21) Appl. No.: 12/317,784

(22) Filed: Dec. 31, 2008

Related U.S. Application Data
(60) Provisional application No. 61/066,865, filed on Feb. 25, 2008.

(51) Int. Cl. C12N 15/00

(52) U.S. Cl. 435/455

(57) ABSTRACT

A method to divert glucose away from glycolysis is described. The method is based on a planned “glucose sink” by introduction, into tumor cells, of nucleotide sequences from known genes whose products are comprised in lactose synthase enzymatic complex. The method is a process designed to correct, replenish or de novo induce lactose synthesis in cancer cells planned to be achieved by insertion of the cDNA of missing protein products capable of lactose synthesis by an expression vector under a strong promoter/enhancer. Intended introduction of the said enzyme expression system by a vector into the cancer cells is by transfection in culture for research. For therapeutic use introduction in vivo is intended by transvection/infection via nanotechnological delivery or viral delivery when delivery systems will be authorized. The best embodiment of this method is in medicine/veterinary where it may require a diagnostic step that defines in individual tumors, especially in mammary tumors, the quantitative and qualitative expression of endogenous genes that enable lactose synthesis, including mainly β4GalT1, α-lactalbumin, and galactose-generating glucose-epimerase, originating preferably from the same species in the case of human subjects. Expression deficiency, deletions, mutations or absence of one of these genes is a guide for induction, correction (if mutated) or replenishment.

### BACKGROUND OF THE INVENTION

This invention is about treatment of cancer cells that are resistant to therapy. Treatment of cancer patients is conventionally directed towards proliferating cells using various strategies via chemotherapy, radiotherapy, hormonal, or immunotherapy. The targeted molecules in general are those involved in the DNA replication machinery, proteins controlling the cell cycle, and the signal transduction pathways that regulate these processes and also affect cell death/survival regulators and cell surface tumor-antigens. In spite of all these methods one of the unsolved problems in the treatment is inability of the therapists to completely eradicate all the cancer cells. This problem may stem from the fact that many targeted molecules operate in intracellular networks that provide replacement options for each or most inhibited key molecules involved in maintaining a particular function, and in our case maintenance of the cancerous state, that results in selection (by the therapy itself) of resistant cells. Hypothetically this is referred to as redundancy that is resulting from options for cross talk between signaling or metabolic pathways to circumvent a mutation or a therapy-induced block in a diseased pathway. Redundancy may also result from evolutionary gene duplications that see to it that in most of the cases there will be a replacement for a therapy-inhibited gene or molecule (Kafri et al. 2005; Kafri et al. 2008; Kafri et al. 2006).

Examples of redundancy are resistance developed in tumors by circumventing inhibitors of EGFR (epidermal growth factor receptor) (Thomson et al. 2008) and inhibitors of cell cycle molecules (L'Italien et al. 2006). A situation has been observed in which glycolysis, one of the two main cellular pathways for ATP synthesis (the other being mitochondrial ATP synthesis), is the most important basis for life maintenance and that tumor cell survival depends on either of these two ATP sources. About 80 years ago Otto Warburg noticed that tumor cells have a lower respiration quotient based tumor cells usage of less oxygen than the normal tissues while they increased glycolysis even in the presence of sufficient oxygen (aerobic-glycolysis) and has summarized these results 3 decades later (Warburg 1956). Upon advancement in understanding of the respiration process, electron transport and oxidative phosphorylation in the mitochondria, Warburg hypothesized that carcinogenesis results from mitochondrial damage to respiration (Warburg 1956). Criticism raised against the Warburg's hypothesis (Weinhouse 1956) doesn't diminish the importance of the Warburg effect itself, i.e. the increased glycolysis in tumor cells. It has been shown that glycolytic enzymes are more abundant in tumor cells (Shaw 2006). Further more, it has been shown that in tumor cells resistant to treatment glycolytic enzymes were more abundant than before treatment (Klein et al. 2006) and in treatment sensitive cells at least one glycolytic enzyme (GAPDH) behaved as being mutated. This means that in resistant tumors glycolysis is the “last stronghold” against therapy and therefore a week point should be searched in glycolysis, an idea that has been taken advantage of by others. Patent No WO/1998/03677/A1 claimed inhibition of LDH-A which targets the distal end of the glycolytic pathway. Patent No AU/2003/204075/B2 claimed phosphofructokinase2 (at the proximal end of glycolysis) to be used as a diagnostic means for cancer and its inhibition as a target in therapy. Both are not discriminatory between resistant tumors and normal tissues. Since the residual cells are usually the ones that kill the patients, the present invention fulfills an existing need. U.S. Pat. No. 5,876,714 and references therein are about post translational modification by glycosylation. U.S. Pat. No. 5,876,714 claimed the enzymatic process related to EC-2.4.1.144 as one involved in the metastatic spread of cancer cells, this is because it represents the process in which glycosyltransferases generate N-acetyllactosaminyl moieties and other glycosylation moieties at the tumor cell surface. It differs from the present invention because it determines properties unrelated to the present invention invention. The EC-2.4.1.144 enzymatic activity clearly differs from EC-2.4.1.22 (the target of the present invention) which is carried out by expression of the heterodimer between β4GalT1 and Lalba (α-lactalbumin) in its normal state of lactation, where it results in lactose synthesis rather than lactosaminyl moiety. The preparation of oligomeric-lactalbumin against cancer claimed by Svanborg et al (U.S. Pat. No. 7,053,185B1) derives from its manufacturing from milk. Svanborg also claimed in U.S. Pat. No. 7,270,822B2 forms of α-lactalbumin to be used directly on cell surfaces to induce apoptosis where the protein is called HAMLET. As such it is basically a modified human α-lactalbumin optionally used (with a cofactor) on the surface of papilloma tumors. This is clearly different from the present invention. In addition Svanborg also claims in U.S. Pat. No. 6,808,930B1/WO9927967A1 diagnosing and killing cancer cells using protein forms of α-lactalbumin fused to a nuclear localization molecule. Cooper et al (U.S. Pat. No. 5,852,224A) claim (vectors) expression systems to generate human α-lactalbumin by expressing its gene under mammary-specific regulatory sequences in bovine or other animal cells to obtain animal milk that contains human α-lactalbumin. Bleck and Bremel invention (U.S. Pat. No. 5,530,177) is about generating transgenic mice expressing bovine lactalbumin under improved regulatory sequences from the respective mammary gland. The present invention fulfills a clear diagnostic and therapeutic need. It is clearly discernible from the above mentioned patents, as it consists of a vector using a promoter with a general ability to be transcription wise active in animal cells to enhance expression of silent or deranged lactose synthase in therapy-resistant cancer cells. The invention is based on the principle of a “glucose sink” competing against, and temporarily depriving, the glycolytic pathway of its main substrate, (glucose) to generate a metabolic crisis selectively in resistant cancer cells.

### SPECIFICATION OF THE INVENTION

This application is about a procedure to treat resistant cancer cells in a selective manner, especially breast cancer cells, (but not excluding other cancers) that are resistant to conventional treatment (resistant to chemotherapy, radiotherapy or immunotherapy). In order to show that the product is not obvious or trivial it is necessary to stress that this invented product for treatment relates to the principle of aerobic hyperglycolysis in tumors. It is based on the premises that resistant cancer cells in general (not only breast cancer cells) are characterized by the following features, but not excluding other features: 1) Complete or partial failure of their mitochondrions to synthesize ATP, a state in which the phosphorylation activity of their oxidative-phosphorylation is failing (Rodriguez-Enriquez et al. 2008), (specifically the ADP phosphorylation and not necessarily the oxidative activity). 2) The glycolytic pathway is intact and can be hyperactive (Shaw 2006). 3) The third feature is the basis for the invention; presence of an option, preferentially tissue-specific, for an “energy-metabolism sink”, i.e. an identified endogenous tissue-specific mechanism optionally (but not necessarily) expressed in the original normal tissue that normally shares substrate/s with the glycolytic pathway. This inherent feature enables competition with or antagonism against the glycolytic pathway that may be tolerated by normal cells (or chemotherapy sensitive cells) but not by cells that show the above first two features. It may also involve wasting of the synthesized ATP on processes that are not necessarily household activities for the tissue that gives rise to the diseased cells. This approach excludes the use of modified glycolytic metabolites that are not tumor tissue-specific such as deoxyglucose. It differs from targeting LDH (Patent No WO/1998/03677/A1) and differs from inhibition of iPFK-2 (Patent No AU/2003/204075/B2) that, although targeting glycolysis, is not specific to resistant tumors and could affect normal tissues.

This application, for the sake of enablement, provides one main working example for the above mechanism in a prototype of a resistant breast cancer cell line model. In this model treatment, in culture with the invented principle mechanism, has killed residual/resistant cells. The resistant cells were the residual cells that resulted from a chemical treatment, a concentration of AG592 that kills 70-90% of MCF7 cells (a notorious human breast cancer cell line). Treatment, by this invention, consists of reconstituting by transfection with (or by any other mode of functional activation of) polynucleotide/s encoding the component/s of lactose synthase in the said exemplified model. This shall be the gene-exons or mRNA or cDNA that, although typical to this tissue upon specific stimulation, is/are either not expressed or expressed in a mutated form in the cancer cells. In the case of MCF7 it is the α-lactalbumin constituent of the enzyme that appears mutated, in other breast cancer-cells it might be the β-4galactosyltransferase (β-4GalT1 coding exons) constituent of the lactose synthase, or perhaps the glucose epimerase or any other protein on which synthesis of lactose is dependent. In the case of α-lactalbumin mutation, an alternative treatment might be the transfection with a specific inhibitor of transcription or translation of this mutated constituent (shown by me). This is in order to free the intact β-4GalT1 (enzymatic partner of α-lactalbumin) from its dominant-negative mutated catalytic α-lactalbumin enhancing partner. The example herein provided includes also transfection with an antisense of α-lactalbumin, which had an effect similar to the sense-form of the said cDNA, as long as it was not mixed with the sense orientation cDNA. The preferred embodiment of the invention example is in the transfer of the non-expressed or wrongly expressed encoding polynucleotide responsible for lactose synthase inactivity. In the case of lack of transcriptional/translational-expression of lactose synthase (or its upstream proteins), priority is claimed for the use of a compound that on it face value is designated to induce the particular transcription and translation of the silent gene or its failed transcripts, the purpose being to enable activity of lactose synthase. This invention should be distinguished from the patent for mass production manufacturing α-lactalbumin, as a protein in any form for any purpose (U.S. Pat. No. 7,053,185, PCT/IB98/01919). It should also be distinguished from the manufacturing of glycosyltransferases (U.S. Pat. No. 5,876,714). The main idea of this invention is in killing the therapy-resistant cancer cells by harnessing their inherent ability to express (also, if exogenously provoked by the therapist) proteins that antagonize or compete with their own glycolysis in a tumor status-specific (resistant Vs sensitive) fashion. The invented products are distinguished from the constructs presented in U.S. Pat. No. 5,852,224A, by virtue of the used coding cDNA, the construct principle, and the vector activation sequences. The distinction results from different purposes of the present invention Vs their inventions. It does not intend to cover the initial interaction of extracted/purified or manufactured protein components, relevant to the said invention, with the diseased cell surface. It rather intends to induce the specific enzymatic components designed to compete with the glycolytic pathway to indirectly (but resistant-tumor-specifically) inhibit its ATP synthesis, and waste prior available ATP.

### Introduction to the Glucose Sink Procedure:

Many breast cancer cells constitutively express Stat3 that in normal mammary epithelium is involved in winning in contrast to Stat5 that participates in preparing the mammary gland for lactation (Clarkson et al. 2006; Robinson et al. 2007). Both Stats are activated downstream to receptors that are targets for the lactogenic hormone prolactin. Previous studies (Klein et al 2006) showed that a putative Stat3 inhibitor (AG592, Calbiochem) was most efficient in killing the MCF7 cell line, (ic₅₀=0.4 μM), with 3 μM 80-90% of the cells were killed, and 10-20% were resistant to killing. 2Dgels revealed in the resistant cells increased abundance of glycolytic enzymes and low endoplasmic reticulum chaperons (calreticulin and prolyl disulfate isomerase). One GAPDH tryptic-digest fragment (V¹⁶³-K¹⁸⁶) showed on MS-MS an abnormally shorter fragment in the (pre 48 h exposure) AG592-sensitive population. In terms of molecular mass this particular peptide tryptic fragment was consistent with the GAPDH sequence I¹⁷⁰-K¹⁸⁶+adenine that did not occur in the resistant cells. This indicated (although indirectly) the presence of a mutation that substituted the normal Gly¹⁶⁹ codon (preceding I¹⁷⁰) by Arg or Lys, thus adding an additional tryptic cleavage site only in the cell population that was sensitive to AG592. These results suggested to the principle author that deranged glycolytic function makes these tumor cells sensitive to treatment. Contrarily, abundant and intact glycolytic pathway enzymes support resistant tumor cells survival under AG592 treatment.

B. Y. Klein went on to survey several features relevant in resistant alveolar mammary gland cancer. 1) He examined whether AG592-resistant MCF7 cells can be further propagated under AG592. 2) Does a fall in estrogen receptor mRNA [a marker for resistant MCF7 cells (Baker et al. 1992; Madsen et al. 1995)] occur under AG592 selection? 3) Is lactose synthase expressed in MCF7 cells?

The findings were that amplification of the resistant cells and re-exposure to AG592 still killed cells, although it killed a much lower percentage. This indicated selection for resistance to AG592 on one hand however, there is probably continuous generation of sensitivity to treatment under a possible genomic instability among the resistant cells. Real time PCR (TaqMan) for MCF7 cDNA showed that HGPRT cDNA is a stable internal control for the relative quantitation assay, of one cycle treated (48 h), vehicle treated and untreated cells. However, cells passed for one month several times in 3 μM AG592 didn't show any HGPRT signal, thus AG592 has an additional effect. Estrogen receptor cDNA fell by ⅓ and β4galactosyltransferase-1 fell by 9/10 in the HGPRT-negative AG592-resistant cells. RT-PCR for α-lactalbumin did not show any signal in any of the above MCF7 samples, despite its protein immunoreactivity detected in western blots. Interestingly, in addition to immunoblots, fluorescent anti α-lactalbumin antibodies detected intracellular and membranous fluorescence, in contrast to the above mentioned negative RT-PCR, indicating that MCF7 cells express, at the protein level, α-lactalbumin antigenicity but with a possible mutation at the mRNA level that was perhaps responsible for the lack of TaqMan PCR signal. This model suggests that a tissue-specific induction of glycolytic pathway inhibitors could be an important addition to killing therapy resistant tumor cells. Direct inhibition of the glycolytic pathway in tumors per se is not the novelty in this patent application, because it has been claimed by two previous applications. The novelty in the present application is a designed induction of lactose synthase expression by the tumor cells that should compete against glycolysis for its glucose substrate and affect only the resistant cells. To enable demonstration of the present invention validity an inducible vector was used. The vector, designed to express α-lactalbumin, has been transiently transfected into MCF7 cells, it has killed 10% of the AG592-untreated populations and 20%-33% of the post 48 h AG592-residuals, the later % approximates the transient transfection efficiency for the calcium precipitation method that has been used.

The cDNA components of lactose synthase and their insertion sites in the vector. The human α-lactalbumin cDNA (Lalba) accession No NM_{—}002289 was cloned into the inducible vector pOPRSVI/MCS via SpeI to NotI restriction sites by appending the SpeI nucleotides to the 5′ end of the cDNA such to ensure an ORF (open reading frame). The NotI was appended to the 3′-end, of the nucleotide sequence that is shown below.

5′-ATGAGGTTCTTTGTCCCTCTGTTCCTGGTGGGCATCCTGTTCCCTGC
CATCCTGGCCAAGCAATTCACAAAATGTGAGCTGTCCCAGCTGCTGAAAG
ACATAGATGGTTATGGAGGCATCGCTTTGCCTGAATTGATCTGTACCATG
TTTCACACCAGTGGTTATGACACACAAGCCATAGTTGAAAACAATGAAAG
CACGGAATATGGACTCTTCCAGATCAGTAATAAGCTTTGGTGCAAGAGCA
GCCAGGTCCCTCAGTCAAGGAACATCTGTGACATCTCCTGTGACAAGTTC
CTGGATGATGACATTACTGATGACATAATGTGTGCCAAGAAGATCCTGGA
TATTAAAGGAATTGACTACTGGTTGGCCCATAAAGCCCTCTGCACTGAGA
AGCTGGAACAGTGGCTTTGTGAGAAGTTGTGA-3′

For the antisense positioning of the above nucleotide sequence, (NM_{—}002289) the negative strand had its 5′ end appended to SpeI and its 3′ end appended to NotI such to obtain the exact opposite orientation within the same vector. (ORF is not necessary for anti-sense functional inhibition of translation).

The 5′-end of the human β4-galactosyltransferase cDNA (GalT1, accession No NM_{—}001497) was appended to a KpnI and the 3′end to XhoI restriction site hexa-nucleotides. This fragment was cloned into the multiple cloning site of pOPRSVI/MCS such to obtain an ORF of the sense orientation for the sequence depicted below.

5′ATGAGGCTTCGGGAGCCGCTCCTGAGCGGCAGCGCCGCGATGCCAGGC
GCGTCCCTACAGCGGGCCTGCCGCCTGCTCGTGGCCGTCTGCGCTCTGCA
CCTTGGCGTCACCCTCGTTTACTACCTGGCTGGCCGCGACCTGAGCCGCC
TGCCCCAACTGGTCGGAGTCTCCACACCGCTGCAGGGCGGCTCGAACAGT
GCCGCCGCCATCGGGCAGTCCTCCGGGGAGCTCCGGACCGGAGGGGCCCG
GCCGCCGCCTCCTCTAGGCGCCTCCTCCCAGCCGCGCCCGGGTGGCGACT
CCAGCCCAGTCGTGGATTCTGGCCCTGGCCCCGCTAGCAACTTGACCTCG
GTCCCAGTGCCCCACACCACCGCACTGTCGCTGCCCGCCTGCCCTGAGGA
GTCCCCGCTGCTTGTGGGCCCCATGCTGATTGAGTTTAACATGCCTGTGG
ACCTGGAGCTCGTGGCAAAGCAGAACCCAAATGTGAAGATGGGCGGCCGC
TATGCCCCCAGGGACTGCGTCTCTCCTCACAAGGTGGCCATCATCATTCC
ATTCCGCAACCGGCAGGAGCACCTCAAGTACTGGCTATATTATTTGCACC
CAGTCCTGCAGCGCCAGCAGCTGGACTATGGCATCTATGTTATCAACCAG
GCGGGAGACACTATATTCAATCGTGCTAAGCTCCTCAATGTTGGCTTTCA
AGAAGCCTTGAAGGACTATGACTACACCTGCTTTGTGTTTAGTGACGTGG
ACCTCATTCCAATGAATGACCATAATGCGTACAGGTGTTTTTCACAGCCA
CGGCACATTTCCGTTGCAATGGATAAGTTTGGATTCAGCCTACCTTATGT
TCAGTATTTTGGAGGTGTCTCTGCTCTAAGTAAACAACAGTTTCTAACCA
TCAATGGATTTCCTAATAATTATTGGGGCTGGGGAGGAGAAGATGATGAC
ATTTTTAACAGATTAGTTTTTAGAGGCATGTCTATATCTCGCCCAAATGC
TGTGGTCGGGAGGTGTCGCATGATCCGCCACTCAAGAGACAAGAAAAATG
AACCCAATCCTCAGAGGTTTGACCGAATTGCACACACAAAGGAGACAATG
CTCTCTGATGGTTTGAACTCACTCACCTACCAGGTGCTGGATGTACAGAG
ATACCCATTGTATACCCAAATCACAGTGGACATCGGGACACCGAGCTA
G-3′

The negative strand, of the above positively oriented fragment, had its 5′end appended to the KpnI restriction site and its 3′end to the XhoI restriction site. Thus an antisense orientation was obtained by insertion into the respective sites of the same multiple cloning site. The pOPRSVI/MCS and the pCMVLac-I vectors used in this example belong to the LacSwitch II Inducible Mammalian System purchased from Stratagene, and for which specifications are available in the instruction manual Catalog #217450, Revision #083004c, that was readily found in Google search engine. http://64.233.183.104/search?q=cache:_Kidhp91Hg8J:www.stratagene.com/manuals/2 17450.pdf+poprsvi/MCS+stratagene&hl=en&ct=clnk&cd=l&gl=uk&client=firefox-a

Example for operation of a designed glucose sink (or ATP sink) procedure: MCF7 cells constitute the working example in which it is necessary to either correct its endogenous α-lactalbumin by forcefully make the cells activate an exogenous proper cDNA, or activate an anti-sense cDNA. The antisense is expected to free the endogenous normal β-4galactosyltransferase-1 (GalT1) protein from its abnormal Lalba (α-lactalbumin) partner, which doesn't provide help, or even inhibits, the lactose-synthase function for which this couple of proteins normally works as one enzyme complex. The experiment below addresses the killing effect by the use of normal Lalba cDNA, it doesn't address the actual synthesis of lactose, for which recruitment of funds could risk the patentability of this invention.

MCF7 breast cancer cell line was seeded in 25 cm² Nunc flasks 10⁶ cells/flask and after 24 h cultures underwent transient transfection using the calcium precipitation method. These were mainly co-transfections of 0.01 mg of the lac repressor expression vector and 0.001 mg of the lac-inhibitable cDNA expression that was subsequently induced on a proper time, using IPTG to lift the lac repression.

1)—One flask was left without transfection.
2)—One flask was mock transfected with calcium phosphate precipitate without DNA.
3)—One flask was transfected with 0.01 mg pCMVlac only, (without its repression target) this vector constitutively expresses the lac-I repressor protein.
4)—One flask was co-transfected by pCMVLac-I+pOPRSVI/MCS−Lalba were Lalba (α-lactalbumin) is designed to be expressed in its sense orientation. The Lalba cDNA is inserted in the multiple cloning site downstream to 2 lac-operator sequences that follow a strong RSV promoter. The RSV promoter is inhibited by the lac repressor protein that binds to the 2 lac-operators, the lac repressor protein is expressed by the co-transfected vector pCMVLac-I, unless IPTG is added to the cells to lift the repression.
5)—One flask was co-transfected with pCMVlac+pOPRSVI/MCS−Lalba+pOPRSVI/MCS−albaL, albaL is the antisense orientation of Lalba.
6)—One flask was co-transfeted with pCMVlac+pOPRSVI/MCS−Lalba and pOPRSVI/MCS−1TlaG which expresses the antisense of endogenous GalT1.

The flasks were washed to remove the transfection solution 6 h after transfection with 10 ml PBS the flasks were then incubated with growth medium (see below) up to 24 h post transfection at which point the cells were trypsinized and seeded in 96-well plates, 5000 cells/well, in Dulbacco modified Eagles medium 4.5 g glucose/liter. The medium was supplemented with 10 ng bovine insulin/ml, penicillin+streptomycin (each 100 microgram/ml), 2 mM glutamine, and 10% fetal calf serum. The medium volume during the first 24 h (2^{nd} 24 h post transfection) was 0.1 ml/well. Note that 16 wells served as blank (A1, B1, C1, D1, E1, F1, G1, H1, A12, B12, C12, D12, E12, F12, G12, H12 were without cells). At 48 h post transfection, each plate was divided into 4 equal quarters to which 0.1 ml of medium of different compositions were added. The 0.1 ml medium added to the upper left quarter (an equal volume to the already present medium) contained ×2 concentration of DMSO (vehicle of AG592) and ethanol (vehicle of IPTG). To the upper right quarter ×2 DMSO and 0.02 mM IPTG were added. These two upper quarters served as the 100% values relative to the lower quarters, that were expected to be diminished by AG592 and designated to become the residual cell base line (lower left) and the response of this base line to the vectors (lower right). To the lower left quarter ×2 AG592 (0.006 mM) and ×2 ethanol (IPTG vehicle) were added. To the lower right quarter ×2 AG592 and ×2 IPTG were added. 96 h after adding these agents the cells were fixed by adding 0.05 ml of 2.5% glutaraldehyde for 10 min. The cultures were now ready for colorimetric cell counting. The glutaraldehyde was washed from the plates with tap water and 0.05 ml of 0.1M borate buffer (pH 8.5) was added followed by 0.1 ml of 0.01% methylene blue W/V (prepared in the same borate buffer) with which the plates were incubated for 1 h at room temp. Following staining excess dye was washed from the plates, plates were dried at room temperature and 0.1 ml of 0.1N HCl/well was added to elute the cell-bound dye that was counted for its optical density on an ELISA reader. The cell counts showed the following results in respect to the above mentioned plate number order:
1) In non-transfected cultures, residual cells that survived treatment with AG592 had 15.8%±2 cells (of their respective 100% controls) that responded to AG592+IPTG (IPTG=inducer of vectors expression) by showing 16.5%±3 of their respective 100% controls. This shows that the non transfected cells that survived the killing by AG592 did not diminish further by the use of IPTG (if anything, their counts slightly, but not significantly, increased).
2) In mock transfected cells (calcium phosphate precipitates without vectors) reference AG592 residuals had 12.5%±1.5 cells (of their respective 100% controls) that responded to AG592+IPTG by showing 15.8%±5 of their respective 100% controls. This shows that the calcium phosphate precipitates per-se did not increase killing above what was caused by AG592 alone upon addition of IPTG (if anything, their counts slightly, but not significantly, increased).
3) In cultures transfected with the pCMVlac-I alone (the vector that expresses the IPTG-sensitive lac repressor protein) showed that reference-AG592 residuals had 14%±1.9 cells (of their respective 100% controls). These cultures responded to AG592+IPTG by showing 17.3%±2 of their respective 100% controls. This shows that transient transfection with pCMVlac-I of cells that survived the killing due to AG592, did not further diminish by the use of IPTG (if anything, their counts increased by 23%, p=0.001 n=20).
4) In co-transfected cultures with pCMVlac-I+pOPRSVI/MCS−Lalba (carrying α-lactalbumin cDNA). The AG592 residual cells in these cultures showed 10.8%±1 of the reference controls and upon expression-induction with IPTG there was a significant (33%, p=0.008, n=20) further decrease in counts as expected. It means that by complementing the endogenously expressed GalT1 with its natural normal partner to become lactose synthase AG592-resistant cells succumb to the transfection at an extent consistent with the efficiency of transient transfection. Had transient transfection efficiency been 100% the projection would have been that 100% of the resistant residuals would have died.
5) In cultures co-transfected with pCMVlac-I+pOPRSVI/MCS−Lalba (carrying α-lactalbumin cDNA)+pOPRSVI/MCS−ablaL (carrying anti-sense oriented α-lactalbumin cDNA). The AG592 residuals in these cultures showed 8.2%±1.8 of the reference controls and upon expression induction with IPTG there was no decrease in counts (as expected, only 9.3%±7.2). This was to show that that when the ablaL (anti-sense of Lalba) was used it inhibited the ability of the sense oriented Lalba to complement the endogenously expressed GalT1 to a full lactose synthase.
6) In cultures co-transfected with pCMVlac-I+pOPRSVI/MCS−Lalba (carrying α-lactalbumin cDNA)+pOPRSVI/MCS−1TlaG (carrying cDNA of an anti-sense oriented β4galactosyltransferase-1). The AG592 residuals in these cultures showed 10.3%±2.7 of the reference controls and upon expression induced with IPTG there was no decrease in counts as expected (only a 10.3%±2.7 decrease). This was to show that similarly to group 5, but here with the other enzyme partner GalT1, its inhibition with the respective anti-sense oriented cDNA (1TlaG) intention to induce failure to express a full-fledged lactose synthase did prevent killing of the AG592-resistant cells.

In an experiment similar to the above, where in similar cultures AG592-residuals cells were co-transfected using pCMV-lac with Lalba anti-sense (ablaL) cDNA it has killed 16.9% of residual cells (p=0.027). Co-transfection with GalT1 cDNA has killed 24.4% of the cells (p=0.0001), and Lalba (sense oriented) cDNA killed 20.3% residual cells (p=0.005). The slightly positive results upon the use of the ablaL antisense alone is interesting. This is because it shows that the α-lactalbumin endogenously expressed in these cancer cells might have a dominant negative effect, due to mutation, by its ability to inhibit the function of its lactose synthase partner (GalT1). It means that inhibiting the endogenous α-lactalbumin expression may weaken the resistance of such tumor cells to therapy.

This is the preferred embodiment of the invention for the study of the glucose sink and ATP sink in therapy resistant tumors in culture, especially in breast cancer cells. It is also optional (as a prophetic example) to raise tumor clones that are selected for expression in 100% of the cells as in stable transfectants but under inducible condition. In non-breast cancer cells the preferred embodiments consists of triple-gene transfection, under the lac-repressor or dual-gene transfection by other inducible means (e.g. Tet⁺ or Tet⁻). For the in vivo cases the preferred embodiment is gene delivery systems developments for gene therapy. Experiments with DNA delivery systems are not advisable to be performed now, because of required grant applications with risk of public disclosure and also because some waiting is needed for ultimate development of delivery systems accepted by relevant authorities.

### REFERENCES

Baker, W. J., V. J. Wiebe, S. K. Koester, V. D. Emshoff, J. U. Maenpaa, G. T. Wurz, et al. (1992). “Monitoring the chemosensitizing effects of toremifene with flow cytometry in estrogen receptor negative multidrug resistant human breast cancer cells.” Breast Cancer Res Treat 24(1): 43-9.
Clarkson, R. W., M. P. Boland, E. A. Kritikou, J. M. Lee, T. C. Freeman, P. G. Tiffen, et al. (2006). “The genes induced by signal transducer and activators of transcription (STAT)3 and STAT5 in mammary epithelial cells define the roles of these STATs in mammary development.” Mol Endocrinol 20(3): 675-85.
Falsone, S. F., B. Gesslbauer, F. Tirk, A. M. Piccinini and A. J. Kungl (2005). “A proteomic snapshot of the human heat shock protein 90 interactome.” FEBS Lett 579(28): 6350-4.
Kafri, R., A. Bar-Even and Y. Pilpel (2005). “Transcription control reprogramming in genetic backup circuits.” Nat Genet 37(3): 295-9.
Kafri, R., O. Dahan, J. Levy and Y. Pilpel (2008). “Preferential protection of protein interaction network hubs in yeast: evolved functionality of genetic redundancy.” Proc Natl Acad Sci USA 105(4): 1243-8.
Kafri, R., M. Levy and Y. Pilpel (2006). “The regulatory utilization of genetic redundancy through responsive backup circuits.” Proc Natl Acad Sci USA 103(31): 11653-8.
Klein, B. Y., S. D. Tachado, H. Koziel and H. K. Avraham (2006). “Protein Changes Typical for Therapy-resistant Cancer Cells Appear in MCF7 Breast Cancer Cultures as Early as One Doubling Time after Chemical Treatment” International Journal of Cancer Research 2(2): 161-75.
L'italien, L., M. Tanudji, L. Russell and X. M. Schebye (2006). “Unmasking the redundancy between Cdk1 and Cdk2 at G2 phase in human cancer cell lines.” Cell Cycle 5(9): 984-93.
Madsen, M. W., B. E. Reiter and A. E. Lykkesfeldt (1995). “Differential expression of estrogen receptor mRNA splice variants in the tamoxifen resistant human breast cancer cell line, MCF-7/TAMR-1 compared to the parental MCF-7 cell line.” Mol Cell Endocrinol 109(2): 197-207.
Robinson, G. W., M. Pacher-Zavisin, B. M. Zhu, A. Yoshimura and L. Hennighausen (2007). “Socs 3 modulates the activity of the transcription factor Stat3 in mammary tissue and controls alveolar homeostasis.” Dev Dyn 236(3): 654-61.
Rodriguez-Enriquez, S., J. C. Gallardo-Perez, A. Aviles-Salas, A. Marin-Hernandez, L. Carreno-Fuentes, V. Maldonado-Lagunas, et al. (2008). “Energy metabolism transition in multi-cellular human tumor spheroids.” J Cell Physiol 216(1): 189-97.
Shaw, R. J. (2006). “Glucose metabolism and cancer.” Curr Opin Cell Biol 18(6): 598-608.
Thomson, S., F. Petti, 1. Sujka-Kwok, D. Epstein and J. D. Haley (2008). “Kinase switching in mesenchymal-like non-small cell lung cancer lines contributes to EGFR inhibitor resistance through pathway redundancy.” Clin Exp Metastasis.
Warburg, O. (1956). “On respiratory impairment in cancer cells.” Science 124(3215): 269-70.
Warburg, O. (1956). “On the origin of cancer cells.” Science 123(3191): 309-14.
Weinhouse, S. (1956). “On respiratory impairment in cancer cells.” Science 124(3215): 267-9.

1) A method to kill populations of tumor cells (comprising the entire animal kingdom) that are resistant to conventional killing methods
wherein a lactose synthase nucleotide sequence component, or components, will be introduced in said tumor cells under a, tissue non-specific, positive transcriptional activation designed to activate transcription of said enzyme nucleotide sequences.
a) A method according to claim-1, for cases of mammary cancer, wherein the lactose synthase nucleotide sequences expression will overcome negative effects of any of the endogenous said enzyme components expression.
b) A method according to claim-1 wherein the expression vector will contain the mammary specific and lactation-specific β-4galactosyltransferase-1 (β4GalT1) nucleotide sequence, designed to enable transcription and synthesis of the said gene protein-product in its functional enzymatic form, regardless of host tissue specificity.
c) A method according to claim-1b wherein the expression vector will contain a nucleotide sequence comprising the coding portion of the cDNA of human α-lactalbumin when the host species is human.


SEQUENCE LISTING (2 sequences)

SEQ ID NO: 1 - DNA, 429 bp, Human
atgaggttctttgtccctctgttcctggtgggcatcctgttccctgccatcctggccaagcaattcacaaaatgtgagctgtcccagctgctgaaagacatagatggttatggaggcatcgctttgcctgaattgatctgtaccatgtttcacaccagtggttatgacacacaagccatagttgaaaacaatgaaagcacggaatatggactcttccagatcagtaataagctttggtgcaagagcagccaggtccctcagtcaaggaacatctgtgacatctcctgtgacaagttcctggatgatgacattactgatgacataatgtgtgccaagaagatcctggatattaaaggaattgactactggttggcccataaagccctctgcactgagaagctggaacagtggctttgtgagaagttgtga

SEQ ID NO: 2 - DNA, 1197 bp, Human
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctag